(19) United States
(12) Patent Application Publication
(10) Pub. No.: US 2014/0170663 A1
(43) Pub. Date: Jun. 19, 2014

(54) METHYLATION SIGNATURE FOR REPLICATIVE SENESCENCE OF CELLS IN CULTURE

(71) Applicants: Wolfgang Wagner, Aachen (DE); Carmen Koch, Aachen (DE); Sylvia Joussen, Eschweiler (DE); Anne Schellenberg, Aachen (DE)

(72) Inventors: Wolfgang Wagner, Aachen (DE); Carmen Koch, Aachen (DE); Sylvia Joussen, Eschweiler (DE); Anne Schellenberg, Aachen (DE)

(73) Assignee: RHEINISCH-WESTFAELISCHE TECHNISCHE HOCHSCHULE AACHEN, AACHEN (DE)

(21) Appl. No.: 14/236,634

(22) PCT Filed: Aug. 6, 2012
(86) PCT No.: PCT/EP2012/065376

§ 371 Date: Feb. 3, 2014

(30) Foreign Application Priority Data
Aug. 4, 2011 (EP) 11176593.9

(51) Int. Cl. C12Q 1/68

(52) U.S. Cl. CPC C12Q 1/6883
USPC 435/6.11; 536/24.33

(57) ABSTRACT

A method for determining a replicative senescence status of a cell includes determining a methylation status of at least one CpG-dinucleotide within a region of at least one of about 50,000 bp upstream and downstream of at least one CpG-dinucleotide selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1. The methylation status of each of the at least one CpG-dinucleotide determined is compared with a reference methylation status of the respective CpG-dinucleotide.

### CROSS REFERENCE TO PRIOR APPLICATIONS

This application is a U.S. National Phase application under 35 U.S.C. §371 of International Application No. PCT/EP2012/065376, filed on Aug. 6, 2012 and which claims benefit to European Patent Application No. 11176593.9, filed on Aug. 4, 2011. The International Application was published in English on Feb. 7, 2013 as WO 2013/017701 A1 under PCT Article 21(2).

### FIELD

The present invention relates to DNA-methylation and its association with cellular aging. The present invention in particular relates to a method and to a kit for determining the replicative senescence status of a cell, wherein the methylation status of at least one of the CpG-dinucleotide is determined and compared with a reference methylation status of the respective CpG-dinucleotide.

### SEQUENCE LISTING

The Sequence Listing associated with this application is filed in electronic form via EFS-Web and is hereby incorporated by reference into this specification in its entirety. The name of the text file containing the Sequence Listing is 270114_Sequence_Listing. The size of the text file is 1,603,346 Bytes, and the text file was created on Jan. 27, 2014.

### BACKGROUND

Cell therapy and tissue engineering raise tremendous expectations with respect to future clinical applications. This is, for example, reflected by an increasing number of clinical studies in this technical field. Mesenchymal stromal cells (MSCs) are of particular interest for clinical applications because they can be isolated from a variety of tissues, and because they comprise a rare population of adult stem cells having the potential of multi-lineage differentiation.

It is necessary to isolate cells for clinical use from at least one of a variety of tissues, for example, from dermis, bone marrow or adipose tissue, and to generate a sufficiently large number of cells by expansion in-vitro, i.e., cell culture. Mammalian cells, however, can be expanded in culture for only a limited number of passages. The cells enter a senescent state and stop proliferation. This phenomenon is not observed in cultures of embryonic stem cells and induced pluripotent stem cells as long as these cells maintain their totipotent or pluripotent state. This difference indicates that this phenomenon is based on a regulated aging process rather than a mere accumulation of cellular defects. Cellular aging is associated with increasing cell size and a flattened cellular morphology. The proliferation rate of the cell and in vitro differentiation of various cell types, such as MSCs, declines with increasing numbers of cell passages. Cells moreover acquire mutations in long-term culture which may result in malignant transformation of affected cells.

The alteration of cellular morphology and functionality during cellular aging has a tremendous impact on the engraftment and success of transplantation when cells are used in clinical therapy. It is therefore important to consider cellular aging and the age of cells as a measure for their quality, especially in the emerging field of cellular therapy. No standards for expanding MSCs in vitro and their use in therapy are, however, yet available. This may be due to the lack of standardized conditions for their isolation and the lack of specific molecular markers.

Commonly used parameters for assessing cellular aging are passage number, cumulative population doubling, and time of in vitro culture. However, these parameters have to be documented very thoroughly throughout culture expansion of the cells. It would otherwise be impossible to determine cellular age. Karyotyping and SNP-arrays are recommended for analyzing cells in culture. These methods are not, however, suitable for finding a malignant subclone in a heterogeneous mixture of cells. Expression of senescence-associated beta-galactosidase can discern cells at their senescent stage, but hardly provides a quantitative measure for cellular aging. Cellular senescence is clearly associated with a loss of telomere integrity, and initial telomere length has been shown to correlate with replicative capacity. Telomere length and telomerase expression varies, however, in different cell types and have not proven as reliable quantitative measure for cellular aging.

There is therefore a need for molecular markers which allow for a reliable assessment of the replicative senecscense of cells. It has recently been discovered that long-term culture of MSCs or fibroblasts is associated with specific epigenetic modifications in DNA-methylation profiles (Bork, S. et al., DNA Methylation Pattern Changes upon Long-Term Culture and Aging of Human Mesenchymal Stromal Cells., Aging Cell 9, pp. 54-63 (2010); Koch, C. et al., Specific Age-associated DNA Methylation Changes in Human Dermal Fibroblasts., PLoS ONE 6, e16679 (2011). It was discovered that the methylation pattern of a variety of genes differed in cells from early passages (P2) and late passages (P8 to P15) in that the methylation status at 27,578 CpG dinucleotides was simultaneously analyzed using a microarray.

### SUMMARY

An aspect of the present invention is to provide markers which are more precise and reliable in assessing the replicative senescence of cells, and which may not only allow for distinguishing cells of an early passage from cells of a late passage, but which show a clear and unequivocal correlation between methylation status and passage number.

In an embodiment, the present invention provides a method for determining a replicative senescence status of a cell which includes determining a methylation status of at least one CpG-dinucleotide within a region of at least one of about 50,000 bp upstream and downstream of at least one CpG-dinucleotide selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1. The methylation status of each of the at least one CpG-dinucleotide determined is compared with a reference methylation status of the respective CpG-dinucleotide.

### BRIEF DESCRIPTION OF THE DRAWINGS

The present invention is described in greater detail below on the basis of embodiments and of the drawings in which:

FIG. 1 shows the alteration in methylation of specific genes in relation to the number of passages of the cells in-vitro;

FIG. 2 shows the GRM7-CpG-site #1, the CASR-CpG-site #1, the PRAMEF2-CpG-site #1, the SELP-CpG-site #1, the CASP14-CpG-site #1, and the KRTAP13-3-CpG-site #1, as well as the adjacent 20 nucleotides upstream and downstream of each of these CpG-sites;

FIG. 3 shows the correlation of predicted passage numbers by means of the methylation status of six CpG-dinucleotides with the real passage number of the cells in-vitro;

FIG. 4 shows the predicted passage number of a variety of primary cells and a variety of cell lines;

FIG. 5 shows the methylation status of CpG-dinucleotides within a region of 50,000 bp upstream and downstream of the GRM7-CpG-site #1, the CASR-CpG-site #1, the PRAMEF2-CpG-site #1, the SELP-CpG-site #1, the CASP14-CpG-site #1, and the KRTAP13-3-CpG-site #1 (respectively referred to as “0”) of human bone marrow cells (hBMCs), wherein each x represents the methylation status of an early passage, and each dot represents the methylation status of a later (presenescent) passage;

FIG. 6 shows the methylation status of CpG-dinucleotides within a region of 5,000 bp upstream and downstream of the GRM7-CpG-site #1, the CASR-CpG-site #1, the PRAMEF2-CpG-site #1 (methylation data of this site were not available on the Illumina 450k chip used here), the SELP-CpG-site #1, and the KRTAP13-3-CpG-site #1 (respectively referred to as “0”), and within a region of 1,000 bp upstream and downstream of the CASP14-CpG-site #1 (also referred to as “0”) of human bone marrow cells (hBMCs), wherein each x represents the methylation status of an early passage, and each dot represents the methylation status of a later (presenescent) passage;

FIG. 7 shows long-term growth curves of culture-expanded cells, where human dermal fibroblasts and mesenchymal stem cells from bone marrow and adipose tissue were expanded in vitro using the indicated supplements. The long-term growth curves of the training data are based on the documentation of culture time and calculation of cumulative population doublings;

FIG. 8 shows Cumulative Population Doublings (cPD) reflected by the DNA methylation level of CpG sites GRM7-CpG-site #1 (CpG1), CASR-CpG-site #1 (CpG2), PRAMEF2-CpG-site #1 (CpG3), SELP-CpG-site #1 (CpG4), CASP14-CpG-site #1 (CpG5), and KRTAP13-3-CpG-site #1 (CpG6). The DNA methylation levels of these six specific CpG sites are plotted against the calculated cumulative Population Doublings of the training (A) and validation data (B). A clear correlation between cPD and methylation status is visible for both datasets. Regression lines, equations and regression coefficients are provided in the figure as well as media and supplements;

FIG. 9 shows predictions of cPD, passage number and days in culture based on the Senescence Signature according to the present invention. Linear regression models on the basis of DNA methylation at the 6 specific CpG sites (GRM7-CpG-site #1, CASR-CpG-site #1, PRAMEF2-CpG-site #1, SELP-CpG-site #1, CASP14-CpG-site #1, and KRTAP13-3-CpG-site #1) are applied to calculate cPD (A), passage numbers (B) or days in culture (C) of the validation data. These predictions are plotted against real cPD, passage numbers and days in culture. Regression coefficients are given in FIG. 8; and

FIG. 10 shows a screenshot of an input mask of a software designed for automated prediction of cPD, passages and days in culture of a given cell preparation. After typing in the corresponding methylation beta-values of the 6 specific CpG sites (GRM7-CpG-site #1, CASR-CpG-site #1, PRAMEF2-CpG-site #1, SELP-CpG-site #1, CASP14-CpG-site #1, and KRTAP13-3-CpG-site #1) and pressing the “Calculate” button, the results are calculated and displayed.

### DETAILED DESCRIPTION

In an embodiment of the present invention, the method comprises the steps of determining the methylation status of least one of the CpG-dinucleotides within a region of about 50,000 bp upstream and/or downstream of at least one of the CpG-dinucleotides selected from the group consisting of GRM7-CpG-site #1 (SEQ ID No. 33), CASR-CpG-site #1 (SEQ ID No. 34), PRAMEF2-CpG-site #1 (SEQ ID No. 35), SELP-CpG-site #1 (SEQ ID No. 36), CASP14-CpG-site #1 (SEQ ID No. 37), and KRTAP13-3-CpG-site #1 (SEQ ID No. 38), and of comparing the methylation status of the CpG-dinucleotide(s) with a reference methylation status for each of the respective CpG-dinucleotide(s). By comparing the methylation status of each of the at least one CpG-dinucleotides investigated with a reference methylation status of the respective CpG-dinucleotide, the replicative senescence status is determined. The term methylation status refers to the level of methylation, i.e., the number of methylated versus non-methylated CpG-dinucleotides of a specific GpG-dinucleotide. The multiple cells can, for example, be analysed for their methylation status. The methylation status of least one of the CpG-dinucleotides within a region of about 50,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 of multiple cells, i.e., for multiple corresponding DNA molecules, is thus determined, and compared with the reference methylation status of the respective CpG-dinucleotide, whereby the replicative senescence status is determined. The reference methylation status for a specific CpG-dinucleotide can, for example, be an empirically-determined methylation level representing a correlation between the methylation level of the CpG-dinucleotide and one or more of the duration cells spent in in-vitro culture, the number of passages cells went through, the cumulative population doubling, and the days of in-vitro culture of the cells.

The CpG-dinucleotides within a region of about 50,000 bp upstream and/or downstream of at least one of the CpG-dinucleotides selected from the group consisting of GRM7-CpG-site #1 (SEQ ID No. 33), CASR-CpG-site #1 (SEQ ID No. 34), PRAMEF2-CpG-site #1 (SEQ ID No. 35), SELP-CpG-site #1 (SEQ ID No. 36), CASP14-CpG-site #1 (SEQ ID No. 37), and KRTAP13-3-CpG-site #1 (SEQ ID No. 38) are all suitable to determine the senescence status of different cell populations (e.g., fibroblasts or MSCs cultured under various conditions and with different methods).

In an embodiment of the present invention, a method for identifying a cell culture which is suitable for therapeutic use is provided which comprises the steps of determining the methylation status, i.e., the status of methylated versus non-methylated versions of corresponding DNA molecules, by determining the methylation status of least one of the CpG-dinucleotides within a region of about 50,000 basepairs (bp) upstream and/or downstream of at least one of the CpG-dinucleotides selected from the group consisting of GRM7-CpG-site #1 (SEQ ID No. 33), CASR-CpG-site #1 (SEQ ID No. 34), PRAMEF2-CpG-site #1 (SEQ ID No. 35), SELP-CpG-site #1 (SEQ ID No. 36), CASP14-CpG-site #1 (SEQ ID No. 37), and KRTAP13-3-CpG-site #1 (SEQ ID No. 38) for multiple corresponding DNA molecules, and of comparing the methylation status of each of the at least one CpG-dinucleotides investigated with a reference methylation status of the respective CpG-dinucleotide, whereby the replicative senescence status is determined.

In an embodiment of the present invention, a use of at least one of the DNA molecules is provided comprising at least one CpG dinucleotide within a region of about 50,000 bp upstream and/or downstream of at least one of the CpG-dinucleotides selected from the group consisting of GRM7-CpG-site #1 (SEQ ID No. 33), CASR-CpG-site #1 (SEQ ID No. 34), PRAMEF2-CpG-site #1 (SEQ ID No. 35), SELP-CpG-site #1 (SEQ ID No. 36), CASP14-CpG-site #1 (SEQ ID No. 37), and KRTAP13-3-CpG-site #1 (SEQ ID No. 38) for determining the replicative senescence status of a cell.

In an embodiment of the present invention, a use of at least one of the DNA molecules is provided comprising at least one CpG dinucleotide within a region of about 50,000 bp upstream and/or downstream of at least one of the CpG-dinucleotides selected from the group consisting of GRM7-CpG-site #1 (SEQ ID No. 33), CASR-CpG-site #1 (SEQ ID No. 34), PRAMEF2-CpG-site #1 (SEQ ID No. 35), SELP-CpG-site #1 (SEQ ID No. 36), CASP14-CpG-site #1 (SEQ ID No. 37), and KRTAP13-3-CpG-site #1 (SEQ ID No. 38) for identifying a cell culture which is suitable for therapeutic uses.

In an embodiment of the present invention, a kit is provided for determining the replicative senescence status of a cell. In an embodiment of the present invention, a kit is provided for identifying a cell culture which is suitable for therapeutic uses.

In an embodiment of the present invention, the methylation status of the CpG-dinucleotide linearly correlates with the replicative senescence status of the cell. The CpG-dinucleotides within a region of about 50,000 bp upstream and/or downstream of at least one of the CpG-dinucleotides selected from the group consisting of GRM7-CpG-site #1 (SEQ ID No. 33), CASR-CpG-site #1 (SEQ ID No. 34), PRAMEF2-CpG-site #1 (SEQ ID No. 35), SELP-CpG-site #1 (SEQ ID No. 36), CASP14-CpG-site #1 (SEQ ID No. 37), and KRTAP13-3-CpG-site #1 (SEQ ID No. 38) are continuously methylated or demethylated with increasing senescence of the cells, i.e., a straight proportional change of the methylation status of these CpG-dinucleotides can be observed during proceeding senescence of the cells.

In an embodiment of the present invention, the methylation status of at least one of the CpG-dinucleotides within a region of about 40,000 bp upstream and/or downstream, for example, of about 30,000 bp upstream and/or downstream, for example, of about 20,000 bp upstream and/or downstream, for example, of about 10,000 bp upstream and/or downstream, for example, of about 5,000 bp upstream and/or downstream, for example, of about 3,000 bp upstream and/or downstream, or, for example, of about 1,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1, is determined.

In an embodiment of the present invention, the replicative senescence status can, for example, be determined in that the methylation status is determined for at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules.

In an preferred embodiment of the present invention, the cell can, for example, have been freshly isolated from a donor.

In embodiment of the present invention, the cell can, for example, have been cultured for some time.

In an embodiment of the present invention, the cell can, for example, have been selected from the group consisting of stromal cells and induced pluripotent stem cells.

In an embodiment of the present invention, the methylation status of two, three, four, five or all of the six CpG-dinucleotides from different DNA molecules which are selected from the group consisting of DNA molecules comprising at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 is determined.

In an embodiment of the present invention, the methylation status can, for example, be determined by one or more methods selected from the group consisting of methylation specific PCR, COBRA-Assay, methylation-specific restriction pattern analysis, CHIP-sequencing, methyl-CAP-sequencing, and sequence analysis of bisulfite-treated DNA.

The present invention further includes the use of at least one nucleic acid molecule for determining the replicative senescence status of a cell according to the method according to the present invention, wherein the nucleic acid molecule comprises at least one nucleotide sequence selected from the group consisting of:
a) a nucleotide sequence comprising at least one of the CpG-dinucleotides within a region of about 50,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1,
b) a nucleotide sequence which differs from the nucleotide sequence of a) by replacement of at most 10% of the nucleotides, but for the CpG-dinucleotide, and
c) a nucleotide sequence which corresponds to the complementary strand of the nucleotide sequence of a) or b).

The present invention also includes the use of at least one nucleic acid molecule for identifying a cell culture which is suitable for therapeutic uses, wherein the nucleic acid molecule comprises at least one nucleotide sequence selected from the group consisting of:
a) a nucleotide sequence comprising at least one of the CpG-dinucleotides within a region of about 50,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1,
b) a nucleotide sequence which differs from the nucleotide sequence of a) by replacement of at most 10% of the nucleotides, but for said CpG-dinucleotide, and
c) a nucleotide sequence which corresponds to the complementary strand of the nucleotide sequence of a) or b).

In an embodiment of these uses, at least one of the CpG-dinucleotides within a region of about 40,000 bp upstream and/or downstream, for example, of about 30,000 bp upstream and/or downstream, for example, of about 20,000 bp upstream and/or downstream, for example, of about 10,000 bp upstream and/or downstream, for example, of about 5,000 bp upstream and/or downstream, for example, of about 3,000 bp upstream and/or downstream, or, for example, of about 1,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 is used.

In an embodiment of these uses, at least one of the CpG-dinucleotides is selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

In an embodiment, the present invention provides a kit for determining the replicative senescence status of a cell, the kit comprising at least one oligonucleotide primer for amplifying and/or analyzing at least one of the CpG-dinucleotides within a region of about 50,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

In an embodiment, the present invention provides a kit for identifying a cell culture which is suitable for therapeutic uses, the kit comprising at least one oligonucleotide primer for amplifying and/or analyzing at least one of the CpG-dinucleotides within a region of about 50,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

In an embodiment of the present invention, the kits comprise at least one oligonucleotide primer for amplifying and/or analyzing at least one of the CpG-dinucleotides within a region of about 40,000 bp upstream and/or downstream, for example, of about 30,000 bp upstream and/or downstream, for example, of about 20,000 bp upstream and/or downstream, for example, of about 10,000 bp upstream and/or downstream, for example, of about 5,000 bp upstream and/or downstream, for example, of about 3,000 bp upstream and/or downstream, for example, of about 1,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

In an embodiment of the present invention, these kits can, for example, comprise at least one oligonucleotide primer for amplifying and/or analyzing at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

In an embodiment of the present invention, the at least one oligonucleotide primer can for example, be selected from the group consisting of SEQ ID NO: 7 to SEQ ID NO: 24.

In an embodiment of the present invention, the kits further can, for example, further comprise at least one reaction buffer and/or reagents for at least one method selected from the group consisting of PCR-amplification, bisulfite-conversion of DNA, DNA-sequencing, preferably DNA-pyrosequencing, and COBRA-assay.

In an embodiment of the present invention, a computer-readable medium can, for example, be provided which has stored computer-executable instructions for causing a computer to perform a method for determining the replicative senescence status of a cell according to the method described above, comprising:
inputting at least one value of the determined methylation status of at least one of the CpG-dinucleotides within a region of about 50,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1,
comparing the value of the determined methylation status with stored data representing a correlation between the methylation status of said CpG-dinucleotide and the replicative senescence status of the cell, and
displaying the replicative senescence status of the cell.

In an embodiment of the present invention, the stored data can, for example, comprise at least one linear regression equation.

The term “replicative senescence status” refers to the potential of a cell to undergo further cell divisions and to differentiate into different cell types. The replicative senescence status of a cell depends on the duration of the cell in in-vitro culture, on the culture conditions, on the number of passages, and on the age of the donor. A cell having a less advanced replicative senescence status has a higher differentiation potential and a higher proliferation potential then a cell having a more advanced replicative senescence status. Hence, when referring to a method to determine the replicative senescence status of a cell, it is intended and suitable for determining any one or more of the duration said cells spent in in-vitro culture, the number of passages said cells went through, the cumulative population doubling, and the days of in-vitro culture of the cells.

These and other aspects of the present invention will be apparent from and elucidated with reference to the exemplary embodiments and to the Figures.

In an embodiment of the present invention, a method for determining the replicative senescence status of a cell is provided. The method comprises the steps of determining the methylation status, i.e., the status of methylated versus non-methylated versions of corresponding DNA molecules, by determining the methylation status of least one of the CpG-dinucleotides within a region of about 100,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules, and of comparing the methylation status of each of the at least one CpG-dinucleotides investigated with a reference methylation status of the respective CpG-dinucleotide, whereby the replicative senescence status is determined.

In an embodiment of the present invention, the method can, for example, comprise determining the methylation status by determining the methylation status of least one of the CpG-dinucleotides within a region of about 50,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules. The method can, for example, comprise determining the methylation status by determining the methylation status of at least one of the CpG-dinucleotides within a region of about 40,000 bp upstream and/or downstream, of about 30,000 bp upstream and/or downstream, of about 20,000 bp upstream and/or downstream, of about 10,000 bp upstream and/or downstream, of about 5,000 bp upstream and/or downstream, of about 3,000 bp upstream and/or downstream, and/or, of about 1,000 bp upstream and/or downstream, of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules. The method can, for example, comprise determining the methylation status by determining the methylation status of least one of the CpG-dinucleotides selected from the group consisting of the CpG-dinucleotides of the GRM7 gene, the CASR gene, the PRAMEF2 gene, the SELP gene, the CASP14 gene, and the KRTAP13-3 gene. The method can, for example, comprise determining the methylation status by determining the methylation status of least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

The region of about 100,000 bp upstream and downstream of CpG-dinucleotide GRM7-CpG-site #1 is represented by SEQ ID No. 33. The region of about 100,000 bp upstream and downstream of CpG-dinucleotide CASR-CpG-site #1 is represented by SEQ ID No. 34. The region of about 100,000 bp upstream and downstream of CpG-dinucleotide PRAMEF2-CpG-site #1 is represented by SEQ ID No. 35. The region of about 100,000 bp upstream and downstream of CpG-dinucleotide SELP-CpG-site #1 is represented by SEQ ID No. 36. The region of about 100,000 bp upstream and downstream of CpG-dinucleotide CASP14-CpG-site #1 is represented by SEQ ID No. 37. The region of about 100,000 bp upstream and downstream of CpG-dinucleotide KRTAP13-3-CpG-site #1 is represented by SEQ ID No. 38. It is understood that the respective SEQ ID Nos: 33 to 38 also include the regions of about 80,000 bp, 60,000 bp, 50,000 bp, 40,000 bg, 30,000 bp, 20,000 bp, 10,000 bp, 5,000 bp, 3,000 bp, and 1,000 bp upstream and downstream of each of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

The cells may be cells that have been freshly isolated from a donor. This embodiment may, for example, permit monitoring the replicative senescence status of cells that were previously implanted for therapeutic use. It is possible to distinguish between native cells of the patient and foreign or native cells that were implanted, i.e., cultured for some time after being isolated from their donor, and before being implanted into the patient. In an embodiment of the method according to the present invention, the cells may have been cultured in-vitro for some time. This embodiment of the method allows determining the approximate time the cells have been in culture, and/or the approximate passage number of the cells, and/or the number of population doublings. In an embodiment of the method of the present invention, the cells can, for example, be mesenchymal stromal cells.

In an embodiment of the present invention, the cells can, for example, be induced pluripotent stem cells. Determining the replicative senescence status for induced pluripotent stem cell can be used to quantify the reprogramming efficiency.

In yet an embodiment of the present invention, the cells can, for example, have been frozen at any time and for any period of time before being analyzed. This embodiment is of particular relevance when retained samples of cells that were implanted for therapeutic use are to be analyzed for their replicative senescent status prior to being implanted.

The replicative senescence status of a cell can be determined by determining the methylation status of a single CpG-dinucleotides within a region of about 100,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules. The methylation status of two, three, four, five, or six different CpG-dinucleotides can, for example, be determined, wherein the two, three, four, five, or six CpG-dinucleotides are chosen from different DNA molecules of the group consisting of DNA molecules comprising GRM7-CpG-site #1, CASR-CpG-site #1, PRAMEF2-CpG-site #1, SELP-CpG-site #1, CASP14-CpG-site #1, and KRTAP13-3-CpG-site #1. The methylation status of two, three, four, five, or all six different CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 can, for example, be determined for multiple corresponding DNA. The more genes of this group involved in determining their methylation status, the more precise the determination of their replicative senescence status will be.

In embodiments of the method according to the present invention, the methylation status can, for example, be determined by one or more suitable methods selected from (but not restricted to) the group consisting of methylation specific PCR, sequence analysis of bisulfite-treated DNA, COBRA-Assay, CHIP-Sequencing, Next-Generation Sequencing, Methyl-CAP-sequencing and methylation-specific restriction patterns. MSP can rapidly assess the methylation status of virtually any group of CpG dinucleotides within a CpG island, independent of the use of cloning or methylation-sensitive restriction enzymes. The MSP comprises initial modification of DNA by sodium bisulfite, converting all unmethylated, but not methylated, cytosines to uracil, and subsequent amplification with primers specific for methylated versus unmethylated DNA.

MSP requires very small quantities of DNA, and is sensitive to 0.1% methylated alleles of a given CpG island locus. The methylation status can, for example, be determined by pyrosequencing of bisulfite-treated DNA which allows identifying whether a specific CpG-dinucleotide was methylated or not, and thereby provides an accurate value for the percentage of methylated CpG-dinucleotides of a given CpG-dinucleotide.

The COBRA assay is a quantitative technique to determine DNA methylation levels at specific gene loci in small amounts of genomic DNA. In the COBRA assay, restriction enzyme digestion is used to reveal methylation dependent sequence differences in PCR products of sodium bisulfite-treated DNA. Methylation levels in the original DNA sample are represented by the relative amounts of digested and undigested PCR product in a linearly quantitative fashion across a wide spectrum of DNA methylation levels. The COBRA assay is easy to use and provides quantitative accuracy.

In the COBRA assay, methylation-dependent sequence differences are introduced into the genomic DNA by sodium bisulfite treatment and subsequent PCR amplification of the bisulfite treated DNA. This combination of bisulfite treatment and PCR amplification results in conversion of unmethylated cytosine residues to thymine and of methylated cytosine residues to cytosine. This sequence conversion can lead to methylation dependent creation of new restriction enzyme sites or it can lead to the methylation dependent retention of pre-existing restriction enzyme sites such as, for example, BstUI (CGCG). The primers used in the PCR amplification reaction do not contain CpG dinucleotides so that the amplification step does not discriminate between templates according to their original methylation status. In the mixed population of DNA fragments resulting from said PCR, the fraction that has a newly created or retained restriction site that contains a CpG(s) directly therefore reflects the percentage of DNA methylation at that site in the original genomic DNA.

Notwithstanding that virtually any method for analyzing the methylation status of a given CpG-dinucleotide can be employed for analyzing the methylation status of at least one of the CpG-dinucleotides within a region of about 100,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1, the methylation status can, for example, be determined by a direct sequence analysis of bisulfite-treated DNA.

In an embodiment of the present invention, the methylation status of at least one of the CpG-dinucleotides can, for example, be selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 is determined by pyrosequencing of bisulfite-treated DNA.

In animals, DNA methylation predominantly involves the addition of a methyl group to the carbon-5 position of cytosine residues of the dinucleotide CpG, and is implicated in repression of transcriptional activity. Treatment of DNA with sodium bisulfite converts cytosine residues to uracil, but leaves 5-methylcytosine residues unaffected. Sodium bisulfite treatment thus introduces specific changes in the DNA sequence that depend on the methylation status of individual cytosine residues, yielding single-nucleotide resolution information about the methylation status of a segment of DNA.

In an embodiment of the present invention, methylation of the at least one CpG-dinucleotide can, for example, be determined by sequence analysis of bisulfite treated DNA.

The method of methylation analysis utilizing sequence analysis of bisulfite-treated DNA involves bisulfite treatment of the DNA to be analyzed, PCR amplification of the bisulfite treated DNA, cloning of the amplification product, and standard dideoxynucleotide DNA sequencing of the cloned DNA fragments to directly determine the nucleotides resistant to bisulfite conversion. Primers for PCR amplification can, for example, be designed to be strand-specific but not methylation-specific, i.e., the nucleotide sequence of the primers do not, for example, comprise a sequence corresponding to a nucleotide sequence including a CpG dinucleotide. The preferred primers for PCR amplification thus flank, but do not involve, the methylation site or methylation sites of interest. In an embodiment of the present invention, at least one or both of the forward primer and the reverse primer for PCR amplification may, for example, cover one or more CpG-dinucleotides. To be strand specific and to allow amplification of methylated as well as non-methylated DNA fragments, a mixture of primers may be utilized, wherein the mixture of strand primers consists of primers having a pyrimidine nucleotide (Y) for the cytosine of the CpG dinucleotide within the DNA fragment to be amplified and covered by said strand primer, i.e., a C for amplification of the DNA fragment which is methylated at said C, or a T for amplification of said DNA fragment which is not methylated at the C. The PCR amplification will therefore amplify both methylated and unmethylated sequences, in contrast to methylation-specific PCR. All sites of unmethylated cytosines are displayed as thymines in the resulting amplified sequence of the sense strand, and as adenines in the amplified antisense strand. This method requires cloning of the PCR products prior to sequencing for adequate sensitivity. Nested PCR methods can alternatively be used to enhance the product for sequencing.

Pyrosequencing may also be used to analyze bisulfite-treated DNA without using methylation-specific PCR. Following PCR amplification of the region of interest, pyrosequencing is used to determine the bisulfite-converted sequence of specific CpG sites in the region. The status of C-to-T at individual sites can be determined quantitatively based on the amount of C and T incorporation during the sequence extension.

In an embodiment of the present invention, methylation of the at least one of the genes selected from the group consisting of GRM7, CASR, PRAMEF2, SELP, CASP14, and KRTAP13-3 can, for example, be determined by combined bisulfite restriction analysis (COBRA assay) where the COBRA-assay can be employed.

The method of determining the replicative senescence status of a cell can be used to determine whether a cell culture comprising cells is suitable for therapeutic use. In an embodiment of the present invention, a method is provided for identifying a cell culture which is suitable for therapeutic use. The method comprises the steps of determining the methylation status, i.e., the status of methylated versus non-methylated versions of corresponding DNA molecules, by determining the methylation status of least one of the CpG-dinucleotides within a region of about 100,000 basepairs (bp) upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules, and of comparing the methylation status of each of the at least one CpG-dinucleotides investigated with a reference methylation status of the respective CpG-dinucleotide, whereby the replicative senescence status is determined.

In an embodiment of the present invention, the method comprises determining the methylation status by determining the methylation status of least one of the CpG-dinucleotides within a region of about 50,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules. The method can, for example, comprise determining the methylation status by determining the methylation status of at least one of the CpG-dinucleotides within a region of about 40,000 bp upstream and/or downstream, of about 30,000 bp upstream and/or downstream, of about 20,000 bp upstream and/or downstream, of about 10,000 bp upstream and/or downstream, of about 5,000 bp upstream and/or downstream, of about 3,000 bp upstream and/or downstream, of about 1,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules. The method can, for example, comprise determining the methylation status by determining the methylation status of least one of the CpG-dinucleotides selected from the group consisting of the CpG-dinucleotides of the GRM7 gene, the CASR gene, the PRAMEF2 gene, the SELP gene, the CASP14 gene, and the KRTAP13-3 gene. The method can, for example, comprise determining the methylation status by determining the methylation status of least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

In an embodiment of the method according to the present invention, the cells can, for example, be mesenchymal stromal cells. In embodiment of the method according to the present invention, the cells can, for example, be induced pluripotent stem cells.

The suitability of a cell culture for therapeutic use can be identified by determining the methylation status of a single CpG-dinucleotides within a region of about 50,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules. The methylation status of two, three, four, five, or six different CpG-dinucleotides is determined, wherein the two, three, four, five, or six CpG-dinucleotides are chosen from different DNA molecules of the group consisting of DNA molecules comprising GRM7-CpG-site #1, CASR-CpG-site #1, PRAMEF2-CpG-site #1, SELP-CpG-site #1, CASP14-CpG-site #1, and KRTAP13-3-CpG-site #1. The methylation status of two, three, four, five, or all six different CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 can, for example, be determined for multiple corresponding DNA. The more genes of the group involved in determining the methylation status, the more precise the determination of the replicative senescence status will be.

The methylation status can be determined by one or more suitable methods selected from the group consisting of methylation specific PCR, sequence analysis of bisulfite-treated DNA, CHIP-sequencing, Methyl-CAP-sequencing, Next-Generation-Sequencing, COBRA-Assay, and methylation-specific restriction patterns. The methylation status can, for example, be determined by pyrosequencing bisulfite-treated DNA as described in more detail herein above, which allows identifying whether a specific CpG-dinucleotide was methylated or not, and thereby provides an accurate value for the percentage of methylated CpG-dinucleotides of a given CpG-dinucleotide.

In an embodiment of the present invention, a use of at least one of the DNA molecules is provided comprising at least one CpG dinucleotide within a region of about 100,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for determining the replicative senescence status of a cell. In an embodiment of the present invention, determining the replicative senescence status of induced pluripotent stem cells can, for example, be used to quantify the reprogramming efficiency. In an embodiment of the present invention, a use of at least one of the DNA molecules is provided comprising at least one CpG dinucleotide within a region of about 100,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for identifying a cell culture which is suitable for therapeutic uses.

In an embodiment of the present invention, the use for determining the replicative senescence status of a cell, and the use for identifying a cell culture which is suitable for therapeutic use can, for example, comprise the use of at least one DNA molecule comprising at least one CpG-dinucleotide within a region of about 50,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules. Any one of the uses can, for example, comprise the use of at least one DNA molecule comprising at least one CpG-dinucleotide within a region of about 40,000 bp upstream and/or downstream, of about 30,000 bp upstream and/or downstream, of about 20,000 bp upstream and/or downstream, of about 10,000 bp upstream and/or downstream, of about 5,000 bp upstream and/or downstream, of about 3,000 bp upstream and/or downstream, or, of about 1,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules. The method can, for example, comprise determining the methylation status by determining the methylation status of least one of the CpG-dinucleotides selected from the group consisting of the CpG-dinucleotides of the GRM7 gene, the CASR gene, the PRAMEF2 gene, the SELP gene, the CASP14 gene, and the KRTAP13-3 gene. The method can, for example, comprise determining the methylation status by determining the methylation status of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

GRM7 denotes the human metabotropic glutamate receptor gene, namely, the gene for the isoform b precursor which is located on chromosome 3 at p26.1. GRM7 is registered under gene ID: 2917 in the NCBI Gene database. The first GRM7 transcript variant (SEQ ID No. 25) is registered in the NCBI Gene Bank database under accession no. NM_{—}000844. The second GRM7 transcript variant (SEQ ID NO: 26) is registered in the NCBI Gene Bank database under accession no. NM_{—}181874.

CASR denotes the human calcium-sensing receptor gene which is located on chromosome 3 at q21.1. CASR is registered under gene ID: 846 in the NCBI Gene database. The first CASR transcript variant (SEQ ID No. 27) is registered in the NCBI Gene Bank database under accession no. NM_{—}001178065. The CASR second transcript variant (SEQ ID NO: 28) is registered in the NCBI Gene Bank database under accession no. NM_{—}000388.

PRAMEF2 denotes the human gene for PRAME family member 2 which is located on chromosome 1 at p36.21. PRAMEF2 is registered under gene ID: 65122 in the NCBI Gene database. The PRAMEF2 transcript variant (SEQ ID No. 29) is registered in the NCBI Gene Bank database under accession no. NM_{—}023014.

SELP denotes the human selectin P precursor gene which is located on chromosome 1 at q24.2. SELP is registered under gene ID: 6403 in the NCBI Gene database. The SELP transcript (SEQ ID No. 30) is registered in the NCBI Gene Bank database under accession no. NM_{—}003005.

CASP14 denotes the human gene encoding the caspase 14 precursor. This gene is located on chromosome 19 at p13.12. CASP14 is registered under gene ID: 23581 in the NCBI Gene database. The CASP14 transcript (SEQ ID No. 31) is registered in the NCBI Gene Bank database under accession no. NM_{—}012114.

KRTAP13-3 denotes the human gene for the keratin associated protein 13-3, the gene being located on chromosome 21 at q22.11. KRTAP13-3 is registered under gene ID: 337960 in the NCBI Gene database. The KRTAP13-3 transcript (SEQ ID No. 32) is registered in the NCBI Gene Bank database under accession no. NM_{—}181622.

In an embodiment of the present invention, a kit-of-parts (kit) is provided for determining the replicative senescence status of a cell. In an embodiment of the present invention, a kit is provided for identifying a cell culture which is suitable for therapeutic uses. The kit for determining the replicative senescence status of a cell, and the kit for identifying a cell culture which is suitable for therapeutic uses contain means for determining the methylation status of at least one of the CpG-dinucleotides within a region of about 100,000 basepairs (bp) upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules.

In an embodiment of the present invention, the kits can, for example, comprise parts rendering the kit suitable for determining the methylation status by determining the methylation status of least one of the CpG-dinucleotides within a region of about 50,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules. The kits can, for example, comprise parts for determining the methylation status by determining the methylation status of least one of the CpG-dinucleotides within a region of about 40,000 bp upstream and/or downstream, of about 30,000 bp upstream and/or downstream, of about 20,000 bp upstream and/or downstream, of about 10,000 bp upstream and/or downstream, of about 5,000 bp upstream and/or downstream, of about 3,000 bp upstream and/or downstream, or, of about 1,000 bp upstream and/or downstream of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules. The kits can, for example, comprise parts for determining the methylation status by determining the methylation status of at least one of the CpG-dinucleotides selected from the group consisting of the CpG-dinucleotides of the GRM7 gene, the CASR gene, the PRAMEF2 gene, the SELP gene, the CASP14 gene, and the KRTAP13-3 gene. The kits can, for example, comprise parts for determining the methylation status by determining the methylation status of at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

In an embodiment of the present invention, the kits can, for example, comprise at least one oligonucleotide primer for analyzing at least one of the CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1, the at least one oligonucleotide primer being adapted for amplifying and/or analyzing a nucleic acid molecule comprising at least one of the nucleotide sequences selected from the group consisting of SEQ ID NO: 1 to SEQ ID NO: 6.

In embodiments of the kits of the present invention, the kits can, for example, comprise at least one oligonucleotide primer selected from the group consisting of SEQ ID NO: 7 to SEQ ID NO: 24.

In an embodiment of the present invention, the kits can, for example, further comprise at least one reaction buffer and/or reagents for at least one method selected from the group consisting of PCR-amplification, bisulfite-conversion of DNA, DNA-sequencing, preferably DNA-pyrosequencing, Next-Generation-Sequencing, and COBRA-assay. These embodiments provide the advantage that all or at least almost all buffers and reagents that are necessary for determining the methylation status are provided with the kit.

While the present invention has been illustrated and described in detail in the drawings and the above description, the drawings and the description are to be considered illustrative or exemplary and not restrictive; the present invention is therefore not limited to the disclosed embodiments.

Other variations to the disclosed embodiments can be understood and effected by persons skilled in the art in practicing the claimed present invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed to be limiting as to scope.

### Example 1

### Isolation of Primary Cells

All samples were taken after written consent and have been specifically approved by local ethic committees on the use of human subjects. Fibroblasts were isolated from dermis (permit number of ethics committee: #EK173/07, Aachen). MSC-AT were isolated from subcutaneous adipose tissue derived from surgical interventions (#EK173/07, Aachen). MSC-BM were isolated from bone marrow aspirations from the iliac crest of healthy donors for allogeneic transplantation or from the caput femoris after hip fracture (#EK128/09, Aachen; #076/2007 and #348/2004, Heidelberg).

### Culture Conditions and Long-Term Growth Curves

Standard culture medium consisted of DMEM medium (PAA, Cölbe, Germany; 1 g/L glucose) with L-glutamine (PAA), penicillin/streptomycin (PAA) supplemented with either 10% fetal calf serum (FCS; Biochrom, Berlin, Germany) or 10% human platelet lysate. Alternatively, a medium consisting of 58% DMEM-LG (Cambrex, Apen, Germany), 40% MCDB201 (Sigma, Deisenhofen, Germany), 2% FCS (Stemcell Technologies, Vancouver, Canada), 2 mM L-glutamine, 100 U/ml Pen/Strep (Gibco, Eggenstein, Germany), 1% insulin transferrin selenium, 1% linoleic acid bovine serum albumin, 10 nM dexamethasone, 0.1 mM L-ascorbic-acid-2-phosphate (all from Sigma) supplemented with platelet derived growth factor (PDGF) and epidermal growth factor (EGF; both 10 ng/ml, R&D Systems, Wiesbaden, Germany) was used as described before.

Cells were always harvested by trypsinisation upon 80% confluent growth, counted with a Neubauer chamber (Brand, Wertheim, Germany) or with a CASY cell counter (Schärfe System, Reutlingen, Germany) and re-seeded at a density of either 5,000 cells/cm² (fibroblasts and MSC-AT) or 10,000 cells/cm² (MSC-AT and MSC-BM). Cell Population doublings per passage (PDP) and cumulative population doublings (cPD) were calculated as described before.

### Quality Control of Cell Preparations

Surface marker expression was analyzed on a FACS canto II (Becton Dickinson Biosciences [BD], Heidelberg, Germany) upon staining with the following antibodies as described before: CD14-allophycocyanin (APC, clone M5E2, BD), CD29-phycoerythrin (PE, clone MAR4, BD), CD31-PE (clone WM59, BD), CD34-APC (clone 8G12, BD), CD45-APC (clone HI30, BD), CD73-PE (clone AD2, BD), CD90-APC (clone 5E10, BD) and CD105-fluorescein isothiocyanate (FITC, clone MEM-226, ImmunoTools, Friesoythe, Germany). Osteogenic, adipogenic and chondrogenic differentiation potential of fibroblasts, MSC-AT and MSC-BM was determined as described before.

### Senescence Tests Based on the Lysosomal Compartment

Expression of pH dependent senescence associated β-galactosidase (SA-β-gal) activity was simultaneously analyzed at different passages using the SA-β-gal staining kit (Cell Signaling Technology, Boston, Mass.) or by flow cytometry with the fluorogenic substrate 5-dodecanoylaminofluorescein di-beta-D-galactopyranoside (C12FDG). Lysosomal and mitochondrial content was analyzed upon 45 min staining of living cells with LysoTracker Red DND-99 (75 nM) and MitoTracker Green FM (100 nM, both Invitrogen/Molecular Probes, Eugene, Oreg., USA). Fluorescence was detected with a FACS Canto II or a Leica DM IL LED fluorescence microscope (Leica, Wetzlar, Germany).

### DNA Isolation and Bisulfite Conversion

Genomic DNA was isolated from 10⁶ cells using the QIAGEN DNA Blood Midi-Kit. DNA quality was assessed with a NanoDrop ND-1000 spectrometer (NanoDrop Technologies, Wilmington, USA) and gel electrophoresis. 600 ng DNA were subsequently bisulfite converted using the EpiTect Bisulfite Kit (Qiagen, Hilden, Germany).

### DNA Methylation Profiling

DNA methylation profiles were analyzed using the HumanMethylation27 Bead Chip according to the manufacturer's instructions (Illumina, San Diego, USA). During hybridization, the DNA molecules anneal to two different bead types with locus-specific DNA oligomers (one corresponds to the methylated (C) and the other to the unmethylated (T) state). Allele-specific primer annealing was followed by single-base extension using DNP- and Biotin-labeled ddNTPs. After extension, the array was fluorescently stained, scanned, and the intensities of the unmethylated and methylated bead types measured. Hybridization and initial data analysis with the BeadStudio Methylation Module were performed at the DKFZ Gene Core Facility in Heidelberg. Raw data of all hybridizations were deposited in NCBIs Gene Expression Omnibus and are accessible through GEO Series accession numbers: GSE17448 (MSC-BM); GSE26519 (MSC-AT); GSE22595 (fibroblasts) and GSE29661 (fibroblasts and MSC-AT).

### Analysis of DNA-Methylation Profiles

Raw data of new datasets and recently published datasets were quantile normalized to minimize chip effects. Principal components analysis (PCA) was calculated with prcomp in R package stats. For selection of relevant CpG sites, Pavlidis Template Matching performed with the MultiExperiment Viewer (MeV, TM4.6) was used. Templates were therefore specified that either corresponded to: 1) the passage numbers of the samples, 2) cPD, or 3) days in culture. The dataset was then searched for matches to the template, based on the Pearson Correlation between the template and methylation values of the data set. For subsequent analysis, only CpG sites with highly significant hyper- or hypo-methylation according to the three corresponding templates (P<10⁻¹¹) were considered. Based on this analysis, six CpG sites (for simplicity, they were termed by their corresponding genes: GRM7, CASR, PRAMEF2, SELP, CASP14 and KRTAP13-3) were selected. Methylation levels of these CpG sites were plotted against passage number for linear regression analysis with EXCEL 2007 (Microsoft). Based on these linear regressions, the state of cellular aging (N) for each of the six CpG sites (i) was calculated by inserting the specific DNA-methylation levels for each gene (β):

Nᵢ=(βᵢ−Aᵢ)/Bᵢ

where A is the Y-axis intercept and B is the slope of the corresponding CpG site in the training group. The mean and standard deviation of the predictions of the six individual CpG sites were subsequently determined as a measure of cellular aging.

### Pyrosequencing

Independent DNA samples of known passage were subsequently bisulfite converted and analyzed by pyrosequencing with regard to the six specific CpG sites. Pyrosequencing was performed at Varionostic GmbH (Ulm, Germany). Primers and sequencing primers are provided in Table 1. Other primers than those disclosed herein may be used for amplifying and/or sequencing the respective nucleic acids.

TABLE 1
Primer for Amplification of Gene
Fragments and Sequencing

| | Gene | | SEQ |
|---|---|---|---|
| | symbol | Nucleotide sequence | ID NO: |
| Forward | GRM7 | TTGGGATTATTGTTGATTT | 7 |
| primer | CASR | TGTAATAGGTATTTGGTTGTAGT | 8 |
| | PRAMEF2 | TTTGAGGGTATTTAGAAGAGAT | 9 |
| | SELP | AGAAGGTAGAAAATTAGTAGAGTT | 10 |
| | CASP14 | TTGGAGATTTAGTGAGATAATA | 11 |
| | KRTAP13-3 | GAGATTTGTTGGAGGTTTAA | 12 |
| Reverse | GRM7 | CCCCTACTACCTACTAAAAATA | 13 |
| primer | CASR | CCCAAACTCTTACTCATTCTA | 14 |
| | PRAMEF2 | TCCCTAACTAACTAACTACTAATC | 15 |
| | SELP | CAACATAAAACTCCATAACTA | 16 |
| | CASP14 | AACAAAACAAATAACCCATATA | 17 |
| | KRTAP13-3 | CCCAATAAAAAACAACTCC | 18 |

| | | | |
|---|---|---|---|
| Sequencing | GRM7 | TACCTACTAAAAATACTCCT | 19 |
| primer | CASR | TTGGTTGTAGTTAGGAA | 20 |
| | PRAMEF2 | TAGAATTTTGTAAAGTGAG | 21 |
| | SELP | AGGTAAAGGTTTAGAAAG | 22 |
| | CASP14 | TATTTTTTTGAGATGGT | 23 |
| | KRTAP13-3 | ATTTTTGTTTGATTATGTA | 24 |

### Quantitative Real-Time PCR Analysis

Expression of the six differentially methylated genes (GRM7, CASR, PRAMEF2, SELP, CASP14 and KRTAP13-3) was analyzed by quantitative real-time PCR (qRT-PCR) using the StepOne™ Instrument (Applied Biosystems [AB], Applera Deutschland GmbH, Darmstadt, Germany). Total RNA was isolated with the miRNeasy Mini Kit (Qiagen, Hilden, Germany) according to the manufacturer's instructions and reversely transcribed. QRT-PCR reactions were performed using Taqman® Gene Express Assays. Gene expression levels were normalized to GAPDH and 18s RNA expression.

Validation with Methylation Profiles

The epigenetic methylation signature was tested on published datasets of other groups. These raw data were generously provided at the public repositories GEO and Array Express. All studies were considered with the HumanMethylation27 BeadChip. None of them provided detailed information on long-term culture. Datasets of either freshly-isolated cells from dermis and epidermis (E-MTAB-202 as described in Gronniger, E. et al., Aging and chronic sun exposure cause distinct epigenetic changes in human skin, PLoS. Genet. 6, e1000971 (2010)); ovarial epithelium (GSE25033 as described in Bauerschlag D O et al., Progression-Free Survival in Ovarian Cancer Is Reflected in Epigenetic DNA Methylation Profiles, Oncology 80, pp. 12-20 (2011)); cervical smear (GSE20080 as described in Teschendorff, A. E. et al., Age-dependent DNA methylation of genes that are suppressed in stem cells is a hallmark of cancer, Genome Res. 20, pp. 440-446 (2010)); cord blood (GSE26683 as described in Novakovic, B. et al., Wide ranging DNA methylation differences of primary trophoblast cell populations and derived-cell lines: implications and opportunities for understanding trophoblast function, Mol. Hum. Reprod. (2011)); and, peripheral blood (GSE25301 as described in Wang, X. et al, Obesity related methylation changes in DNA of peripheral blood leukocytes, BMC. Med. 8, 87 (2010)) or datasets of established cell lines derived from solid cancer (cell lines SW48 and MCF-7; GSE26683 as described in Novakovic, B. et al., Wide ranging DNA methylation differences of primary trophoblast cell populations and derived-cell lines: implications and opportunities for understanding trophoblast function, Mol. Hum. Reprod. (2011)), squamous cell carcinoma (GSE24091), ovarian carcinoma (GSE25033 as described in Bauerschlag, D O et al., Progression-Free Survival in Ovarian Cancer Is Reflected in Epigenetic DNA Methylation Profiles, Oncology 80, pp. 12-20 (2011)), transformed placenta/trophoblast (GSE26683 as described in Novakovic, B. et al., Wide ranging DNA methylation differences of primary trophoblast cell populations and derived-cell lines: implications and opportunities for understanding trophoblast function. Mol. Hum. Reprod. (2011)), multiple myeloma/MGUS (GSE21304 as described in Walker, B. A. et al., Aberrant global methylation patterns affect the molecular pathogenesis and prognosis of multiple myeloma, Blood 117, pp. 553-562 (2011)), lymphoma (GSE26133 as described in Bell, J. T. et al., DNA methylation patterns associate with genetic and gene expression variation in HapMap cell lines, Genome Biol. 12, R10 (2011)) and pluripotent cell lines (GSE24676 as described in Nishino, K. et al., DNA Methylation Dynamics in Human Induced Pluripotent Stem Cells over Time, PLoS. Genet. 7, e1002085 (2011)) were used. For further analysis, only beta-values of the six specific CpG-dinucleotides and used these for the predictive model described above were extracted.

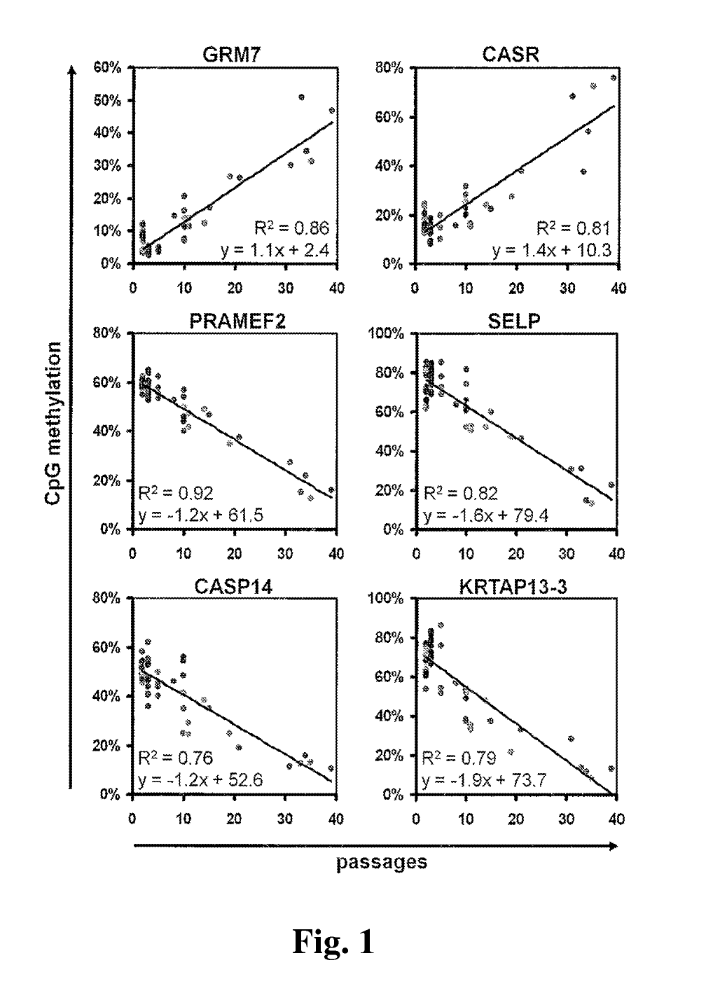

The results of the analysis are briefly summarized in FIG. 1. FIG. 1 illustrates that there is a clear and unequivocal correlation of the number of passages the cells went through and the methylation status of each one of the 6 CpG-dinucleotides selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1. GRM7-CpG-site #1 and CASR-CpG-site #1 become hypermethylated during ongoing passages of in in-vitro culture and thus while becoming replicative senescent, wherein the degree of hypermethylation corresponds to the number of cell passage. In contrast, the CpG-dinucleotides PRAMEF2-CpG-site #1, SELP-CpG-site #1, CASP14-CpG-site #1, and KRTAP13-3-CpG-site #1 become hypomethylated during ongoing passages of in in-vitro culture and thus while becoming replicative senescent, wherein the degree of hypomethylation corresponds to the number of cell passage.

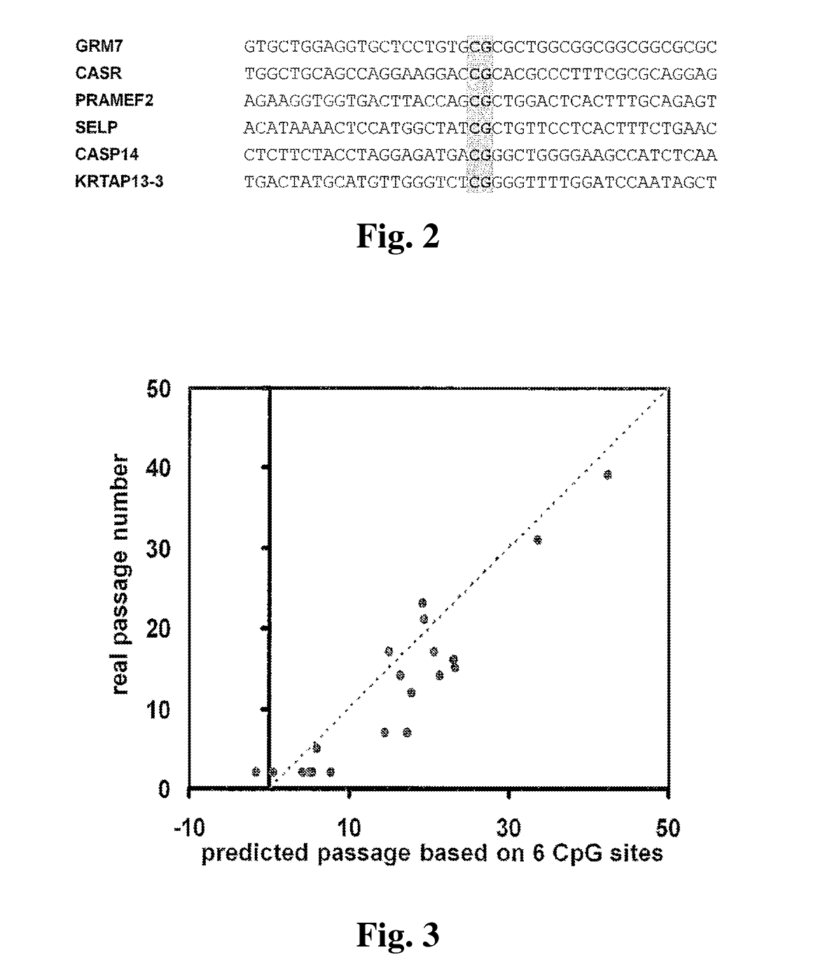

FIG. 2 depicts the genomic sequences of the six CpG-dinucleotides, namely GRM7-CpG-site #1, CASR-CpG-site #1, PRAMEF2-CpG-site #1, SELP-CpG-site #1, CASP14-CpG-site #1, and KRTAP13-3-CpG-site #1, which were used for establishing the epigenetic senescence signature. FIG. 2 shows the respective CpG-dinucleotides and 20 nucleotides of the genomic DNA upstream and 20 nucleotides of the genomic DNA downstream of each of said CpG-dinucleotides. The nucleotide sequences shown in FIG. 2 correspond to the nucleotide sequences set forth in SEQ ID NOs: 1 to 6.

FIG. 3 shows the results where independent cell preparations were used for validation of the senescence-signature by pyrosequencing of the six CpG sites (GRM7-CpG-site #1, CASR-CpG-site #1, PRAMEF2-CpG-site #1, SELP-CpG-site #1, CASP14-CpG-site #1, and KRTAP13-3-CpG-site #1). The beta-values were used for the above-mentioned linear regression models to predict the number of passages. The graph in FIG. 3 clearly shows that the predicted passage number accurately identifies the real passage number of each cell culture.

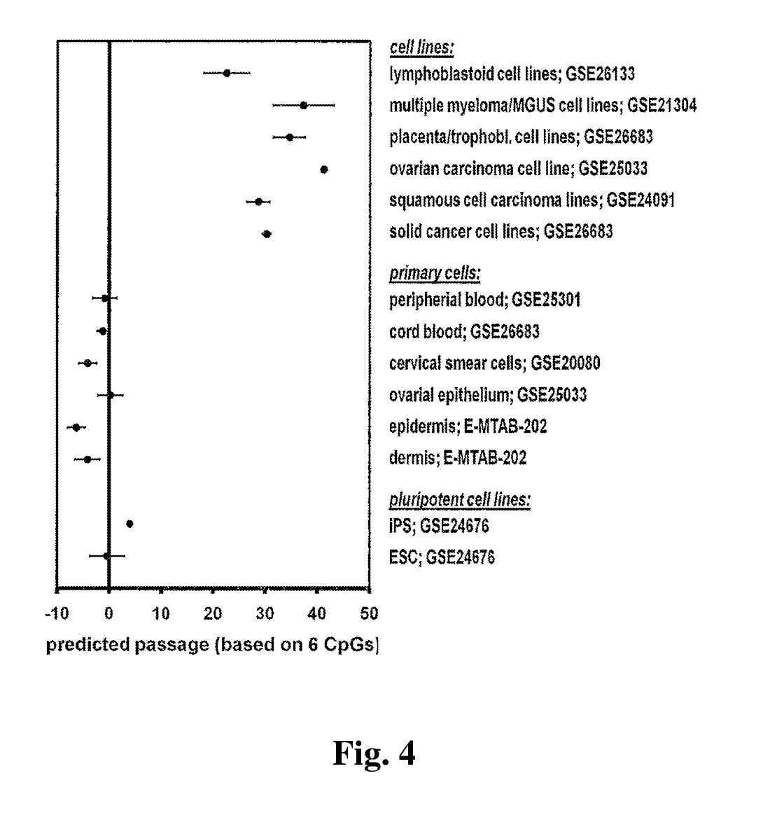

FIG. 4 illustrates that the epigenetic replicative senescence signature is applicable to different cell types and tissues. DNA-methylation datasets were retrieved from public data repositories. DNA-methylation level at the six CpG-dinucleotides (GRM7-CpG-site #1, CASR-CpG-site #1, PRAMEF2-CpG-site #1, SELP-CpG-site #1, CASP14-CpG-site #1, and KRTAP13-3-CpG-site #1) can clearly separate freshly isolated cells and culture expanded cell lines (mean and standard deviation of the different samples are provided).

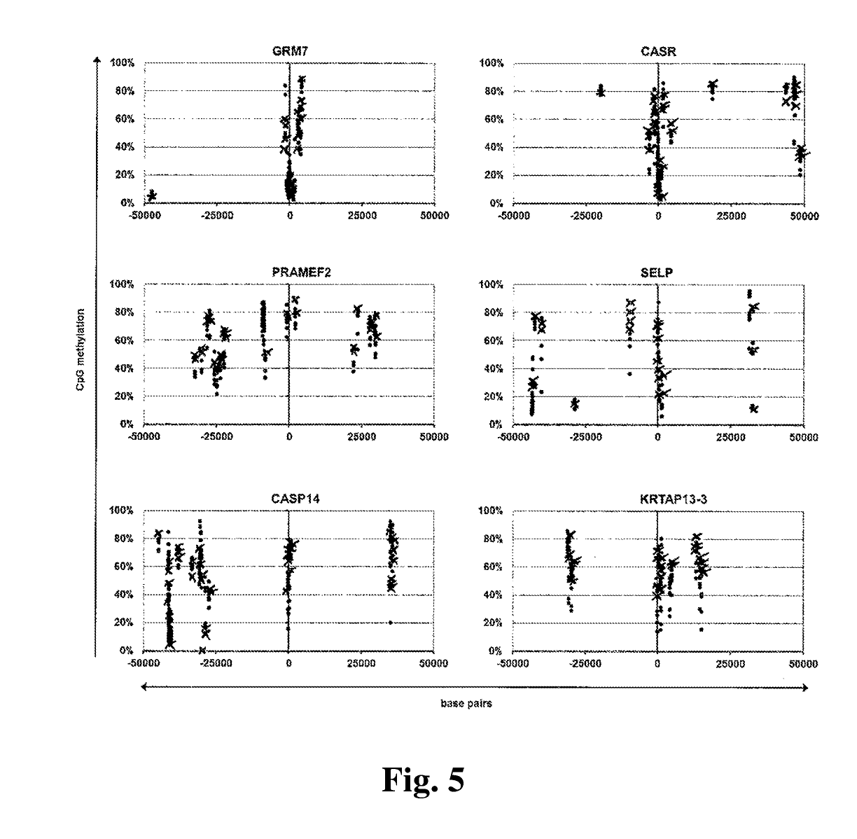

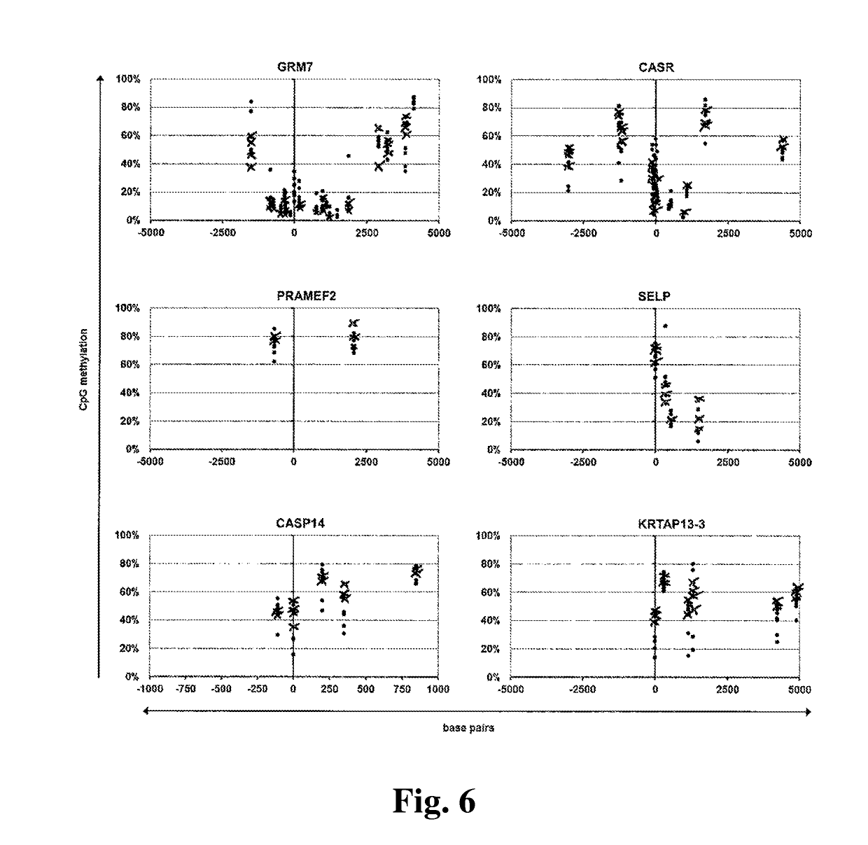

FIGS. 5 and 6 demonstrate that CpG-dinucleotides within a region of about 50,000 bp upstream and/or downstream of at least one of the CpG-dinucleotides selected from the group consisting of GRM7-CpG-site #1 (SEQ ID No. 33), CASR-CpG-site #1 (SEQ ID No. 34), PRAMEF2-CpG-site #1 (SEQ ID No. 35), SELP-CpG-site #1 (SEQ ID No. 36), CASP14-CpG-site #1 (SEQ ID No. 37), and KRTAP13-3-CpG-site #1 (SEQ ID No. 38) are all suitable to determine the senescence status of cell populations. All CpG-sites within the depicted regions are continuously methylated or demethylated with increasing senescence of the cells, i.e., a linear change of the methylation status of these CpG-dinucleotides can be observed during proceeding senescence of the cells.

The data show that the replicative senescence status of cells can be accurately determined by particular epigenetic features.

The present invention provides the advantages that it is not necessary to determine the expression of specific marker genes in order to assess the replicative senescence of a cell or the cells of a cell culture. Comparing the methylation status obtained with a reference methylation status can moreover be done in that the methylation status as determined is inserted into a formula for linear regression. It is thereby possible to determine the replicative senescence status of a cell or of the cells within a cell culture even if no positive or negative control is available. In addition, the methods of the present invention are reliable as the results have been proven to be independent from the persons handling the cell cultures, any growth conditions and the like.

### Example 2

In this approach, all available DNAm data with the Infinium HumanMethylation27 BeadChip were combined. This platform facilitates simultaneous analysis of DNAm at more than 27,000 CpG sites at single base resolution. Those CpG sites which reveal a linear increase in DNAm level over either subsequent passages, days in culture, or cumulative population doublings (cPDs), were subsequently identified and selected. Six specific CpG sites were identified by Pavlidis template matching which fulfilled these parameters. Analysis of DNAm at these CpG sites can conversely be used to predict the state of cellular aging. In the following sections, a step-by-step guidance for the usage of this Epigenetic Senescence Signature is provided.

### Material

### Biological Material

This method is applicable for different cell types, particularly for mesenchymal stromal cells (MSC) and fibroblasts. All cell preparations were isolated after written consent according to the guidelines of the local Ethics Committees. Bone marrow derived MSC (BM-MSC) were isolated from bone marrow aspirates (iliac crest; IC) of healthy donors for allogeneic transplantation (#348/2004, Heidelberg, Germany), from caput femoris (CF) after hip fracture or from tibia plateau (TP; #EK128/09, Aachen, Germany, and #076/2007, Heidelberg). Adipose-tissue derived MSC (AT-MSC) were isolated from lipoaspirates of healthy adult donors (#EK163/07, Aachen). Dermal fibroblasts (D-Fib) were isolated from patients undergoing plastic surgery (#EK173/07, Aachen). The following sections describe exemplarily culture expansion of BM-MSC in medium supplemented with 10% human platelet lysate (hPL).

### Human Platelet Lysate

Human platelet lysate (hPL) is generated by simple freeze-thaw procedures with thrombocyte concentrate units from healthy donors (provided by the blood bank, University Hospital, Aachen).

### Culture Medium and Passaging

1. 500 mL laboratory bottle (Duran, Wertheim, Germany)
2. 500 mL rapid-Filtermax, vacuum filtration unit (TPP, Trasadingen, Switzerland)
3. 2 U/mL Heparin (Ratiopharm, Ulm, Germany)
4. Dulbecco's Modified Eagles Medium-Low Glucose (DMEM-LG; PAA, Pasching, Austria)
5. 2 mM L-glutamine (Sigma-Aldrich, München, Germany)
6. 100 U/mL penicillin/streptomycin (pen/strep; Gibco, Invitrogen, Carlsbad, USA)
7. 0.25% Trypsin-EDTA solution (1×) (Gibco)
8. lx phosphate-buffered saline (PBS) (PAA)
9. Tissue culture flasks 75 cm2 (Nunc Thermo Fisher Scientific, Langenselbold, Germany)
10. Hemocytometer (Neubauer counting chamber, Brand, Wertheim, Germany)
11. 0.4% Trypan Blue solution (Sigma)
12. 15 and 50 mL Falcon tubes (BD)

### DNA Preparation

1. QiaAmp DNA Blood Midi Kit (Qiagen, Hilden Germany)
2. 15 mL Falcon tubes (BD)
3. 0.5 mL Safelock tubes (Sarstedt, Nümbrecht, Germany)
4. Nuclease-Free Water (Qiagen)
5. Waterbath (70° C.)

### DNAm Analysis at Specific CpG Sites

This method is based on the DNAm level at six specific CpG sites. Various methods can be used for this purpose. These CpG sites can easily be selected from DNAm profiles with the Infinium HumanMethylation27 BeadChip. It is expected that it is alternatively possible to determine the DNAm level by MassARRAY assay. In the following sections; the specific analysis of pyrosequencing of bisulfide-treated DNA is used.

### Methods

### Long-Term Culture of Mesenchymal Stomal Cells

1. Add L-glutamine, penicillin/streptomycin, heparin and human platelet lysate (10%) to the DMEM-LG medium to a final volume of 500 mL.
2. Sterile-filter medium and store at 4° C. up to 2 weeks.
3. Seed BM-MSC after isolation at a density of 5,000 cells/cm² into culture flasks.
4. Upon 80% confluence, remove medium and wash cells with PBS twice.
5. Add 1 mL of trypsin-EDTA solution and incubate at 37° C. for 1 minute.
6. After cell detachment, stop trypsination by application of 5 mL culture medium.
7. Transfer solution into 15 mL Falcon tube and centrifuge at 350×g for 7 minutes.
8. Discard supernatant and resuspend pellet in 1 mL culture medium.
9. Count cells in a Neubauer counting chamber after application of Trypan Blue dye for exclusion of dead cells.
10. Re-seed cells at a density of 5,000 cells/cm².
11. Document cell count for calculation of cumulative Population Doublings (see subheading 3.3).

### Calculation of Real Cumulative Population Doublings

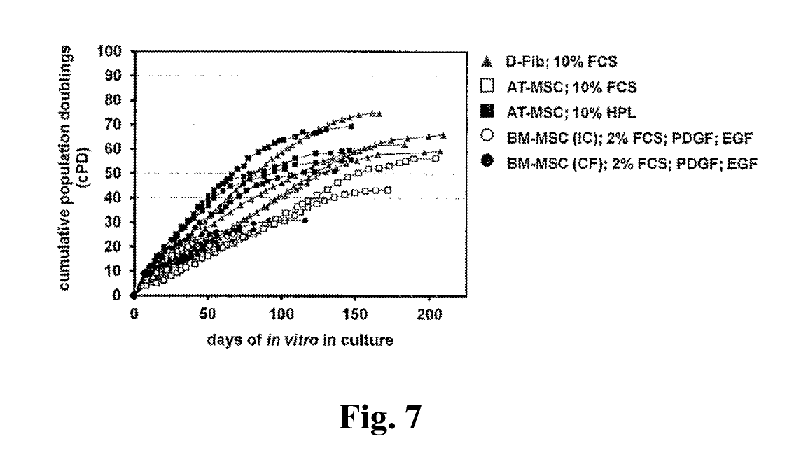

1. Calculate cumulative Population Doublings from the first passage until the destined number of passages by employing the following formula (FIG. 7):

cPD=sum_{i=1 . . . n} log₂(hi/si),
where n is the total number of passages; si is the number of cells seeded at passage i, and hi is the number of cells harvested in passage i.

### DNA Preparation and Bisulfide Conversion

1. Isolate genomic DNA with the QiaAmp DNA Blood Midi Kit following the manufacturer's instructions.
2. Elute DNA in 300 μL Nuclease-Free Water or in the provided Elution Buffer for long-term storage.
3. Assess concentration and quality via photometric measurement and agarose gel electrophoresis.
4. Bisulfide conversion

### DNA Methylation Analysis of the Six Specific CpG Sites

Analysis of the DNAm six specific CpG sites is the prerequisite for usage of the Epigenetic Senescence Signature. If DNAm profiles with the Illumina HumanMethylation27k Chip are available, these can be directly extracted by the IDs (corresponding gene names and sequences are also indicated—the relevant CpG sites are indicated in bold):

| | |
|---|---|
| 1. | GTGCTGGAGGTGCTCCTGTGCGCGCTGGCGGCGGCGGCGCGC |
| | (cg02332525; GRM7) |
| 2. | TGGCTGCAGCCAGGAAGGACCGCACGCCCTTTCGCGCAGGAG |
| | (cg17453778; CASR) |
| 3. | AGAAGGTGGTGACTTACCAGCGCTGGACTCACTTTGCAGAGT |
| | (cg03891191; PRAMEF2) |
| 4. | ACATAAAACTCCATGGCTATCGCTGTTCCTCACTTTCTGAAC |
| | (cg01459453; SELP) |
| 5. | CTCTTCTACCTAGGAGATGACGGGCTGGGGAAGCCATCTCAA |
| | (cg01999333; CASP14) |
| 6. | TGACTATGCATGTTGGGTCTCGGGGTTTTGGATCCAATAGCT |
| | (cg16431978; KRTAP13-3) |

DNAm can be alternatively specifically be determined by pyrosequencing as described above.

Analysis of the Epigenetic Senescence Signature with a Computer-Readable Program

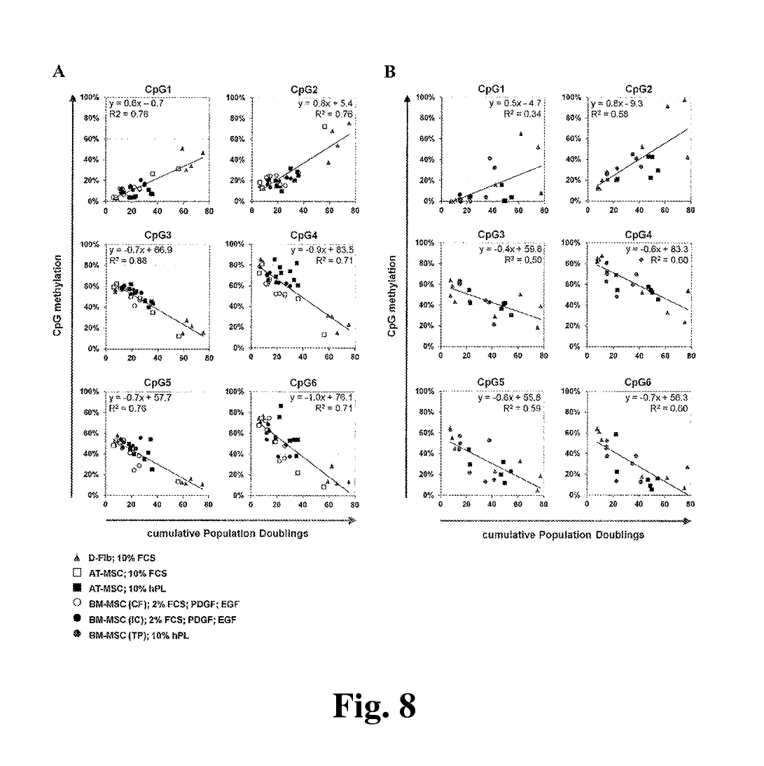

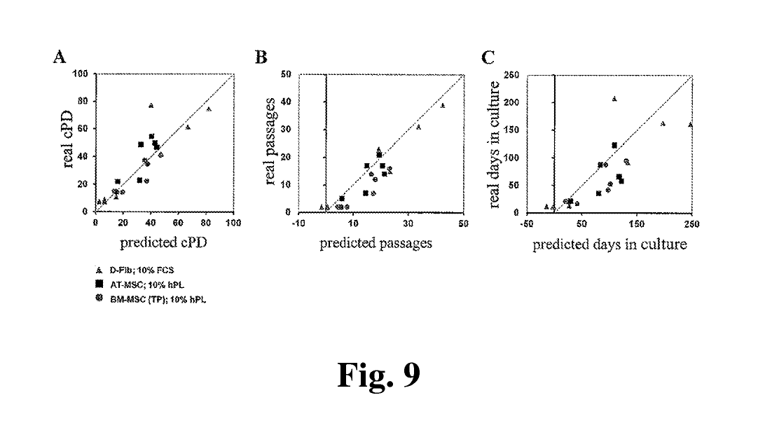

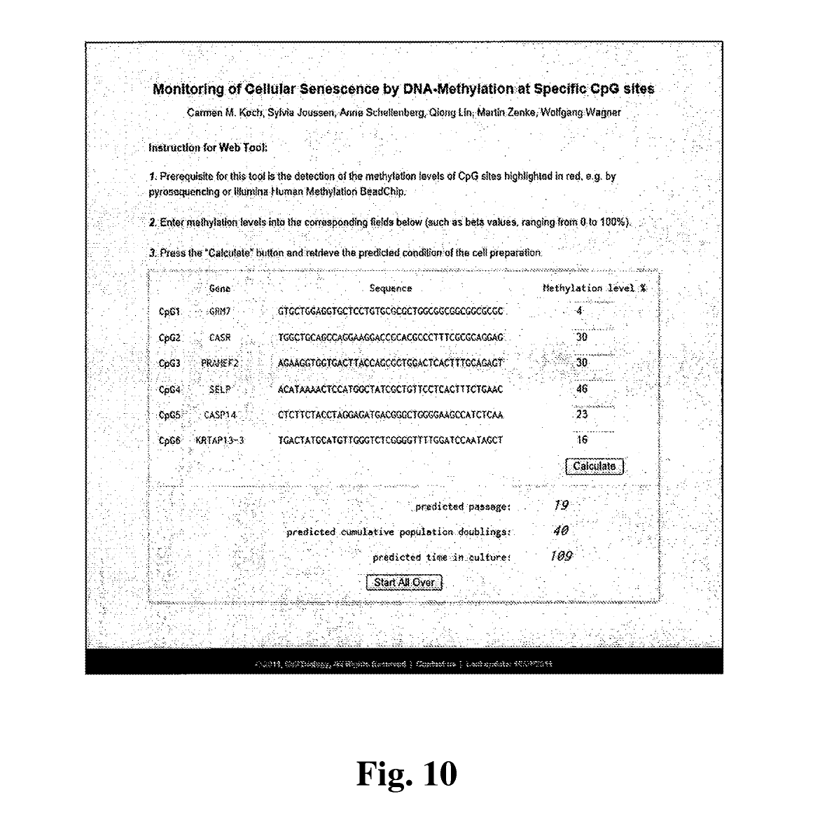

For each CpG site, the corresponding DNAm values need to be inserted in the corresponding linear regression models (FIG. 8). These equations are described in Koch et al., Aging Cell 11, pp. 366-9 (2012). Predictions for cPDs are thereby generated for each individual CpG site and the mean of these is subsequently calculated for the final value (FIG. 9). To make this calculation easier, a software was designed where the DNAm values can easily be integrated. The input mask of this software is shown in FIG. 10.
1. Type in the corresponding beta-values of the 6 CpG sites and press “Calculate” for the predictions.
2. Compare results with the calculated real cPD of the cell preparations.

A multivariate model is alternatively provided which combines the six linear regressions into one equation:

Predicted cPD=45.89+(23.63*cg02332525)+(31.61*cg17453778)+(−53.70*cg03891191)+(14.86*cg01459453)+(−23.94*cg01999333)+(−10.34*cg16431978).

The corresponding beta-values for the six CpG sites must be inserted. This method generates similar results as the above-mentioned method (FIG. 10).

The present invention is not limited to embodiments described herein; reference should be had to the appended claims.

What is claimed is:

1-15. (canceled)

16. A method for determining a replicative senescence status of a cell, the method comprising:
determining a methylation status of at least one CpG-dinucleotide within a region of at least one of about 50,000 bp upstream and downstream of at least one CpG-dinucleotide selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1; and
comparing the methylation status of each of the at least one CpG-dinucleotide determined with a reference methylation status of the respective CpG-dinucleotide.

17. The method as recited in claim 16, wherein the methylation status of the at least one CpG-dinucleotide linearly correlates with a replicative senescence status of the cell.

18. The method as recited in claim 16, wherein the methylation status is determined of the at least one CpG-dinucleotide within a region of at least one of about 40,000 bp upstream and downstream of at least one CpG-dinucleotide selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

19. The method as recited in claim 16, wherein the methylation status is determined for at least one CpG-dinucleotide selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1 for multiple corresponding DNA molecules.

20. The method as recited in claim 16, wherein the cell is selected from stromal cells and induced pluripotent stem cells.

21. The method as recited in claim 16, wherein the methylation status is determined of two, three, four, five or six CpG-dinucleotides from different DNA molecules, which are selected from the group consisting of DNA molecules comprising at least one CpG-dinucleotide selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

22. The method as recited in claim 16, wherein the methylation status is determined by at least one method selected from a methylation-specific PCR, a COBRA-Assay, a methylation-specific restriction pattern analysis, a CHIP-sequencing, a methyl-CAP-sequencing, and a sequence analysis of bisulfite-treated DNA.

23. A method of using at least one nucleic acid molecule to determine a replicative senescence status of a cell as recited in the method as recited in claim 16, wherein the nucleic acid molecule comprises at least one nucleotide sequence selected from the group consisting of:
a first nucleotide sequence comprising at least one CpG-dinucleotide within a region of at least one of about 50,000 bp upstream and downstream of at least one CpG-dinucleotide selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1,
a second nucleotide sequence which differs from the first nucleotide sequence in that at most 10% of the nucleotides of the at least one CpG-dinucleotide are replaced, and
a third nucleotide sequence which corresponds to a complementary strand of the first nucleotide sequence or of the second nucleotide sequence.

24. A method of using at least one nucleic acid molecule to identify a cell culture suitable for a therapeutic use, wherein the nucleic acid molecule comprises at least one nucleotide sequence selected from the group consisting of:
a first nucleotide sequence comprising at least one CpG-dinucleotide within a region of at least one of about 50,000 bp upstream and downstream of at least one CpG-dinucleotide selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1,
a second nucleotide sequence which differs from the first nucleotide sequence in that at most 10% of the nucleotides of the at least one CpG-dinucleotide are replaced, and
a third nucleotide sequence which corresponds to a complementary strand of the first nucleotide sequence or of the second nucleotide sequence.

25. A kit for determining a replicative senescence status of a cell or for identifying a cell culture suitable for a therapeutic use, the kit comprising:
at least one oligonucleotide primer to at least one of amplify and analyze at least one CpG-dinucleotide selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

26. The kit as recited in claim 25, wherein the at least one CpG-dinucleotide is within a region of at least one of about 50,000 bp upstream and downstream of the at least one CpG-dinucleotide selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1.

27. The kit as recited in claim 26, further comprising at least one of a reaction buffer and a reagent for at least one method selected from a PCR-amplification, a bisulfite-conversion of DNA, a DNA-sequencing, a DNA-pyrosequencing, and a COBRA-assay.

28. A computer-readable medium comprising stored computer-executable instructions prompting a computer to perform a method for determining a replicative senescence status of a cell as recited in claim 16, the stored computer-executable instructions including the steps of:
inputting at least one value of a determined methylation status of at least one CpG-dinucleotide within a region of at least one of about 50,000 bp upstream and downstream of at least one CpG-dinucleotide selected from the group consisting of GRM7-CpG-site #1, CASR-CpG-site #1, PRAMEF2-CpG-site #1, SELP-CpG-site #1, CASP14-CpG-site #1, and KRTAP13-3-CpG-site #1;
comparing the at least one value of the determined methylation status with a stored data representing a correlation between a methylation status of the CpG-dinucleotide and a replicative senescence status of the cell; and
displaying the replicative senescence status of the cell.

29. The computer-readable medium as recited in claim 28, wherein the stored data comprises at least one linear regression equation.
